(19) United States
(12) Patent Application Publication
(10) Pub. No.: US 2011/0119779 A1
(43) Pub. Date: May 19, 2011

(54) METHODS FOR SEQUENTIAL REPLACEMENT OF TARGETED REGION BY HOMOLOGOUS RECOMBINATION

(75) Inventors: Hiroaki Shizuya, South Pasadena, CA (US); Manish Singh, Woodland Hills, CA (US); Cecilia Roh, South Pasadena, CA (US)

(73) Assignee: ALIVA BIOPHARMACEUTICALS, INC., Pasadena, CA (US)

(21) Appl. No.: 12/747,406

(22) PCT Filed: Dec. 10, 2008
(86) PCT No.: PCT/US2008/086275

§ 371 Date: Dec. 3, 2010

Related U.S. Application Data
(60) Provisional application No. 61/012,701, filed on Dec. 10, 2007.

(51) Int. Cl. A01K 67/027; C12N 15/85; C12N 15/82; C12N 15/63; C12N 5/10; C12N 15/81; C12N 15/74

(52) U.S. Cl. 800/18; 435/463; 435/468; 435/320.1; 435/325; 800/13; 435/471

(57) ABSTRACT

The invention provides methods and compositions for generating non-human transgenic cells and organisms that are transgenic at one or more gene sequences by separately recombining fragments of a complete gene in temporal sequence. According to the methods of the invention, a set of DNA constructs containing a non-endogenous DNA sequence flanked and/or operably linked at its ends by sequences from the non-human organism are generated by recombination in a bacterial cell, for example, in E. coli. The DNA constructs that are produced can then be introduced into a non-human homologous recombination competent cell where successive cells will contain recombined segments of a target gene, with the ultimate cell in a line containing an endogenous target gene completely replaced by genomic DNA of another species.

### CROSS-REFERENCE TO RELATED APPLICATION

This application claims the benefit under 35 U.S.C. §119(e) of U.S. Provisional Patent Application No. 60/012,701 filed Dec. 10, 2007, and this provisional application is incorporated herein by reference in its entirety.

### BACKGROUND

1. Technical Field

The invention relates generally to methods and compositions for gene targeting by homologous recombination and, more specifically, to methods and constructs for transfer of large DNA sequences.

2. Description of the Related Art

Genetic transfer using recombinant technologies has become a foundation for basic research in the biomedical field as well as a cornerstone in the field of human drug discovery. Genetic transfer using recombinant technologies is the foundation for the development of transgenic organisms in which DNA from one species is inserted and expressed in organisms of a different species. Transgenic organisms are now commonly employed in basic research to study the function of genes and their protein products, the role of genetic mutations in disease and in the pharmaceutical industry for the discovery and development of human protein therapeutics.

While recombinant technologies now allow the physical replacement of relatively small regions of chromosomes in transgenic organisms, it is extremely challenging to replace large DNA sequences, e.g., over 50 kb, in the genome of one species with large DNA sequences from that of another species. The classic alternative is to perform two separate modifications, (1) an inactivation of the endogenous locus to be replaced and (2) a separate introduction of the DNA from the other species into another site in the genome. Often, even the introduction of large pieces of DNA on a separate transgene is laborious and time-consuming, and it yields an unsatisfactory recapitulation of gene function due to position and copy-number effects or the purposeful or accidental deletion of important cis-regulatory elements in the transgene. This inability to replace very large tracts of endogenous DNA with orthologous DNA in a cell or transgenic organism has greatly hindered the study of biological systems in vivo, which depend on the coordinated interaction of multiple genes located in long stretches of DNA. Also, some genes are extremely large. It has also hindered the development of important new therapeutic applications of recombinant technology.

Genes and loci that are prime examples of this challenge are numerous and include, but are not limited to the following examples. Human and mouse immunoglobulins (Ig) consist of two types of polypeptide chains (heavy chains, referred to as H chains and light chains, referred to as either λ or κ chains) all of which are encoded by multiple genes consisting of about one to two million contiguous base pairs that function in a complexly coordinated fashion. Other large and complexly structured and regulated genes that are involved in human disease or have potential therapeutic utility include CD45, phenylalanine hydroxylase, factor VIII, cystic fibrosis transmembrane conductance regulator, NF1, utrophin, T-cell receptors, the major histocompatibility complex, and dystrophin. Other multi-gene families of therapeutic interest, e.g., globin genes, growth hormones, albumins, and Fc gamma receptors, are clustered on chromosomes. If one wanted to study the function of these human genes or other human genes of similar size and complexity in mammalian models such as mouse models, it would be necessary to fully inactivate the orthologous mouse genes and introduce the human genes. Ideally, the mouse genes would be replaced by the human genes in their germline (natural) configuration to faithfully recapitulate the correct timing and levels of expression, both at the transcriptional and post-transcriptional levels. This replacement would preferably be achieved by homologous recombination. Using current technology to replace a very large, i.e., greater than 50 kb, mouse gene or locus with its human counterpart would require a very large series of multiple targeted replacement steps. This approach is cumbersome, time consuming and labor intensive.

To overcome these difficulties, one approach uses flanking regions totaling greater than 20 kb of DNA homologous to the host DNA to allow for insertion of the exogenous DNA into precise locations of the host genome by homologous recombination and therein replacing the corresponding genes of the host (see U.S. Pat. No. 6,586,251). To determine whether replacement of the endogenous DNA with the exogenous DNA occurs, quantitative methods such as quantitative PCR are required. For this, probes to the unmodified host allele are used to detect a reduction in the number of unmodified host alleles after the homologous recombination of the exogenous DNA. Simpler methods that do not require quantitative methods are described herein, which can allow for in situ determination of precise insertion, thus facilitating transfer of large DNA sequences from one species to another.

### BRIEF SUMMARY

The present invention discloses a method for transferring large DNA sequences from the genome of one species to the genome of a different species by separate sequential homologous recombination steps. The present method is simpler than previous approaches, providing for the use of simpler qualitative procedures to assess precise insertion of exogenous DNA into a host genome. Specifically, it allows for detection of one or more markers by another set of one or more markers via marker displacement, thereby differentiating cells containing randomly inserted sequences from those undergoing homologous recombination. This makes the process easier to employ while allowing for precise replacement of large DNA fragments.

In one embodiment, a method of sequentially replacing a non-endogenous DNA sequence across a target non-human DNA sequence is disclosed including: a) contacting a cell that has the target non-human DNA sequence with a first DNA construct and homologously recombining the first DNA construct with the target non-human DNA sequence, where the first DNA construct comprises: i) a first non-endogenous DNA sequence flanked by first and second non-human DNA sequences, and ii) a first selection marker sequence;

b) qualitatively determining the presence of the first selection marker in the contacted cell, thereby identifying a first selection marker positive cell;

c) contacting the first selection marker positive cell with a second DNA construct and homologously recombining the second DNA construct with the recombined target non-human DNA sequence including the first non-endogenous DNA sequence, wherein the second DNA construct comprises, i) a second non-endogenous DNA sequence operably linked to a third non-human DNA sequence, wherein the second non-endogenous DNA sequence homologously recombines with a segment of the first non-endogenous DNA sequence of the recombined target non-human DNA sequence, and the third non-human DNA sequence homologously recombines with non-human DNA sequences distal to the second non-human DNA sequence of the first DNA construct, and ii) a second selection marker sequence, wherein the second selection marker sequence is located within the third non-human DNA sequence, and the first and second selection markers are not the same; and

d) qualitatively determining the presence of the second selection marker in the cell comprising the recombined target non-human DNA sequence of step (c), where the homologous recombination at step (c) removes the first selection marker sequence, thereby identifying a second selection marker positive cell; wherein the target non-human DNA sequence is replaced by the non-endogenous DNA sequence.

A related embodiment further comprises the steps of: e) contacting the second selection marker positive cell with a third DNA construct and homologously recombining the third DNA construct with the recombined target non-human DNA sequence of step (d) that has the first and second non-endogenous DNA sequences, wherein the third DNA construct comprises: i) a third non-endogenous DNA sequence linked to a fourth non-human DNA sequence, wherein the third non-endogenous DNA sequence homologously recombines with a segment of the second non-endogenous DNA sequence of the recombined target non-human DNA sequence, and the fourth non-human DNA sequence homologously recombines with non-human DNA sequences distal to the third non-human DNA sequence of the second DNA construct, and ii) a third selection marker sequence, wherein the third selection marker sequence is located within the fourth non-human DNA sequence; and

f) qualitatively determining the presence of the third selection marker in a population of cells having the recombined target non-human DNA sequence of step (e), where the homologous recombination at step (e) removes the second selection marker sequence, thereby identifying a third selection marker positive cell; wherein the target non-human DNA sequence is replaced by the non-endogenous DNA sequence.

Yet another related embodiment further comprises repeating steps (c)-(f), where each added DNA construct includes: i) a non-endogenous DNA sequence that homologously recombines with a segment of the recombined non-endogenous DNA sequence of the previous DNA construct, a non-human DNA sequence that homologously recombines with non-human DNA sequences distal to the non-endogenous and target non-human DNA sequences of the previously recombined DNA construct, and ii) a selection marker sequence, wherein recombination of the additional DNA construct alternately removes the previous selection marker sequence; and wherein step (g) is repeated until the target non-human DNA sequence is replaced by the non-endogenous DNA sequence. In certain embodiments, the first and third selection marker sequences encode the same selection marker.

In some embodiments of the invention, the sequential replacement occurs in the 3′ to 5′ direction, i.e., the second non-endogenous DNA sequence replaces a portion of the target DNA sequence 5′ of the previously recombined first non-endogenous sequence. In other related embodiments, the sequential replacement extends in the 5′ to 3′ direction, i.e., the second non-endogenous DNA sequence replaces a portion of the target DNA sequence 3′ of the previously recombined first non-endogenous sequence.

In one aspect, each non-human DNA sequence flanking the non-endogenous sequence of the first DNA construct is greater than or equal to 20 kb in length. In another aspect, each non-human DNA sequence flanking the non-endogenous sequence of the first DNA construct is less than about 20 kb in length. In yet another aspect, the non-endogenous sequence is orthologous to the target non-human DNA sequence. In another aspect, the non-endogenous sequence is a human DNA sequence.

In certain embodiments of the invention, the cell is a plant cell. In another embodiment of the invention, the cell is a non-human animal cell. In a related embodiment, the non-human animal cell is a mouse embryonic stem cell.

In another aspect, the selection marker is a fluorescent marker. In other embodiments, the selection marker is a drug resistance marker. Another embodiment of the invention includes a second selection marker that is adjacent to the first selection marker. In certain embodiments, one of the selection markers is a fluorescent marker. In another embodiment, one of the selection markers is a drug resistance marker. In yet another embodiment, one of the selection markers is a fluorescent marker, and the second selection marker is a drug resistance marker.

In another embodiment, a set of constructs for sequentially replacing a non-endogenous DNA sequence across a target non-human DNA sequence is disclosed including a first construct including DNA sequences homologous to target DNA sequences, a selection marker sequence, and cloning vector DNA; and a second DNA construct including a non-endogenous DNA sequence to replace an endogenous target DNA sequence, flanking DNA sequences homologous to endogenous sequences in the target cell, a selection marker sequence, and cloning vector DNA. In a related embodiment, the set of constructs further comprises a third construct including a non-endogenous DNA sequence, a DNA sequence homologous to the target DNA sequence in the cell, a selection marker sequence, and cloning vector DNA. In another related embodiment, the set of constructs contains a fourth DNA construct that includes an exogenous DNA sequence, an endogenous DNA sequence homologous to an endogenous sequence in the target cell, a selection marker sequence, and cloning vector DNA.

In one aspect, the DNA sequences of the first DNA construct of the set serve as substrate sequences for homologous recombination with endogenous DNA sequences present in target cells. In a related aspect, the DNA sequences of the first DNA construct of the set serve as both a substrate sequence for homologous recombination and replacement sequences of DNA in the cells.

In one embodiment of the invention, the selection marker is a fluorescent marker. In another embodiment, the selection marker is a drug resistance marker. In another embodiment, the constructs further comprise a second selection marker.

In another aspect, the selection marker is placed within the coding region of the non-endogenous or non-human DNA sequence. In yet another aspect, the selection marker is placed within the non-coding region of the non-endogenous or non-human DNA sequence.

In another aspect, each DNA construct is cloned in a vector. In a related aspect, the vector is a BAC, YAC or PAC vector.

Certain embodiments disclose a cell containing a transgene produced by the methods of the invention. Another embodiment of the invention provides a non-human animal generated from a cell containing a transgene produced by the methods of the invention. A related embodiment provides a humanized mouse comprising a human transgene produced by the disclosed methods of the invention.

In yet another embodiment of the invention, a method for producing a recombined BAC includes: a) contacting a bacterial cell, wherein the bacterial cell comprises a first BAC, with a second BAC, wherein said first BAC comprises a first non-endogenous DNA sequence, a first bacterial selection marker sequence and a cloning vector DNA sequence; and wherein said second BAC comprises a second non-endogenous DNA sequence, a second bacterial selection marker sequence and a cloning vector DNA sequence; wherein said second non-endogenous DNA sequence comprises an overlapping segment of said first non-endogenous DNA sequence; wherein homologous recombination occurs at said overlapping segment; and

b) qualitatively determining the presence of said first and second bacterial selection markers in the bacterial cell having a recombined non-endogenous DNA sequence, wherein the recombined BAC is produced. A related aspect further comprises resolving said recombined BAC, wherein the overlapping segment is removed from the BAC, thereby generating a resolved BAC.

In one embodiment, the first bacterial selection marker is removed from said recombined BAC. In another embodiment, the second bacterial selection marker is removed from said recombined BAC. In yet another embodiment, the first and second selection markers are removed from said recombined BAC.

In certain embodiments, the resolving step includes homologous recombination. In another embodiment, the resolving step includes a site-specific recombinase. In one embodiment, the site-specific recombinase is Cre. In another embodiment, the site-specific recombinase is flp.

In one embodiment, the first selection marker is a drug resistance marker. In another embodiment, the first selection marker is a fluorescent marker. In another embodiment, the second selection marker is a drug resistance marker. In yet another embodiment, the second selection marker is a fluorescent marker. In a related embodiment, the first and second selection markers are drug resistance markers.

One embodiment of the invention provides a recombined BAC produced according to the methods of the invention. A related embodiment provides a resolved BAC generated according to the methods of the invention.

One embodiment of the invention discloses a set of BACs including a) a first BAC comprising a first non-endogenous DNA sequence, a first selection marker sequence, and a cloning vector DNA sequence; and b) a second BAC comprising a second non-endogenous DNA sequence and a second selection marker sequence, wherein the second non-endogenous DNA sequence comprises an overlapping region of the first non-endogenous DNA sequence, wherein homologous recombination occurs at the overlapping region. In a related embodiment, the first selection marker sequence is a fluorescent marker. In another embodiment, the first selection marker sequence is a drug resistance marker. In another embodiment, the second selection marker is a fluorescent marker. In a related embodiment, the second selection marker is a drug resistance marker.

### BRIEF DESCRIPTION OF THE SEVERAL VIEWS OF THE DRAWINGS

FIG. 1 illustrates four types of DNA constructs for sequentially replacing a non-endogenous DNA sequence across a non-human target DNA sequence. In the illustrated DNA constructs, the non-endogenous DNA sequences are human DNA sequences. In this illustration, construct 1A includes a) DNA sequences homologous to endogenous DNA sequences, b) one or more genes that supply selection markers, and c) cloning vector DNA; construct 1B includes a) a human DNA sequence to replace an endogenous target DNA sequence, b) flanking DNA sequences homologous to endogenous sequences in the cell to be transformed or transfected, c) one or more selection marker genes, and d) cloning vector DNA; constructs 1C and 1D include a) a human DNA sequence, b) a non-human sequence that is homologous to the target sequence, c) one or more selection marker genes, and d) cloning vector DNA. For constructs 1C and 1D, the human sequences are not flanked on one side by non-human sequences, and on the opposite side of the human sequences the human and non-human sequences are joined at adjacent positions. The latter two constructs differ in the relative order of the two sequences (i.e., human or non-human sequence relative to telomere or centromere direction). The order is determined by the direction of consecutive replacement of existing sequences in the cells with replacing DNA sequences.

FIG. 2 illustrates homologous recombination between 1) a DNA construct equivalent to construct 1A (for this illustration, identified as 2A) that has an optional third selection marker (e.g., Yellow Fluorescent Protein [YFP]) in addition to Green Fluorescent Protein (GFP) and G418 and 2) a target mouse chromosome (Mouse Chrom 1). Note the replacement construct is inserted in the same relative centromere to telomere orientation as the target gene.

FIG. 3 illustrates homologous recombination between 1) a DNA construct equivalent to DNA construct 1B (for this illustration, identified as 2B) and the target mouse chromosome from the recombination steps depicted in

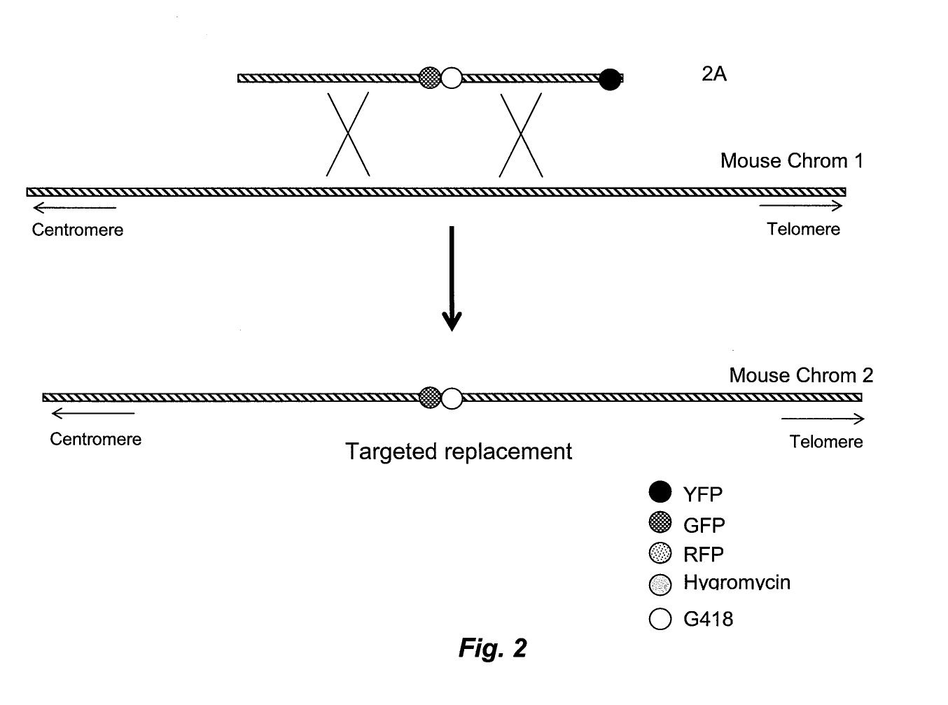

FIG. 2 (Mouse Chrom 2). Upon recombination, the GFP and G418 markers are replaced by Red Fluorescent Protein (RFP) and hygromycin. Again, the replacement construct is inserted in the same relative centromere to telomere orientation. Also note that the YFP is not inserted into the target mouse chromosome and serves as a negative selection marker.

FIG. 4 illustrates homologous recombination between 1) a DNA construct equivalent to construct 1B (for this illustration, identified as 2B) that has an optional third selection marker (YFP) in addition to RFP and G418 and 2) a target mouse chromosome (Mouse Chrom 1). Upon recombination, the resulting mouse chromosome (Mouse Chrom 3) contains the RFP and G418 selection markers.

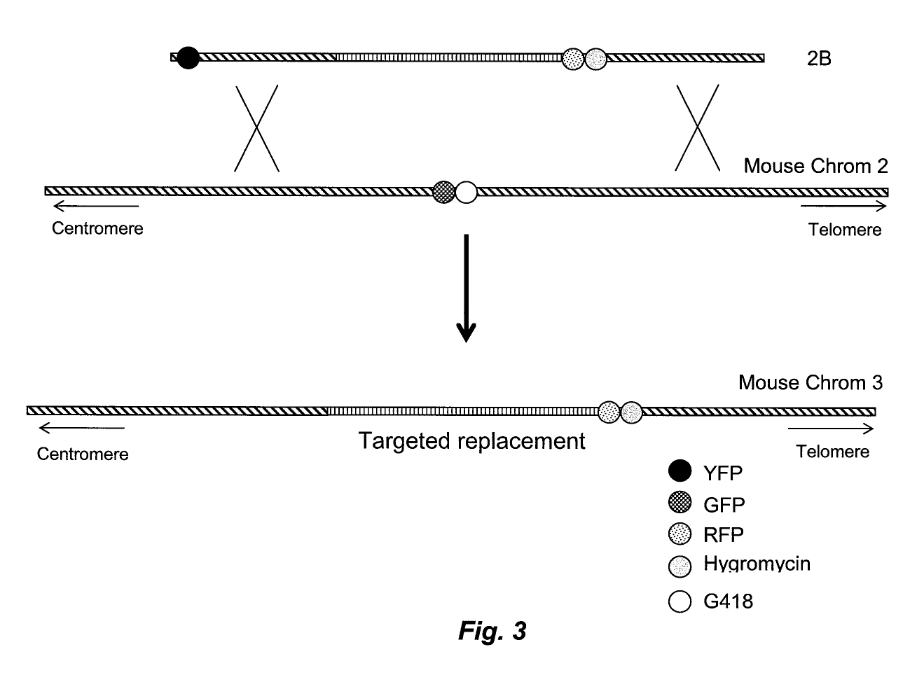

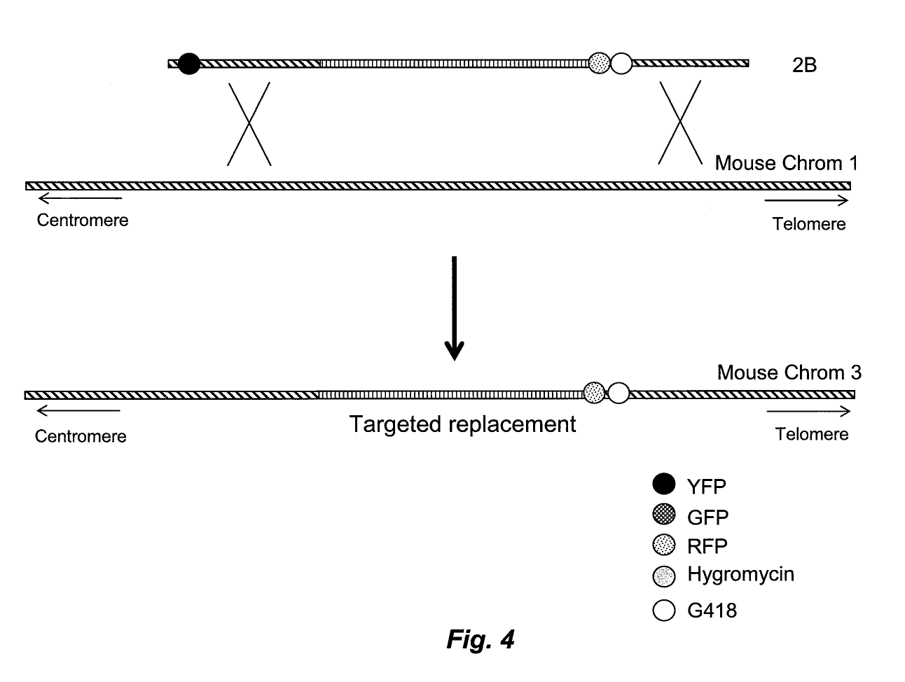

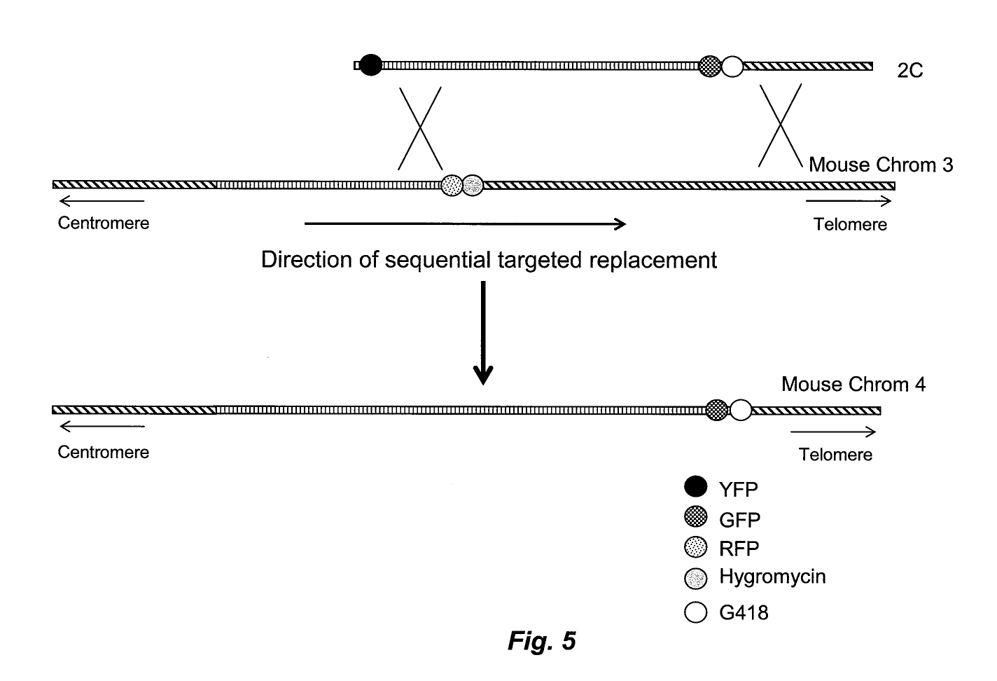

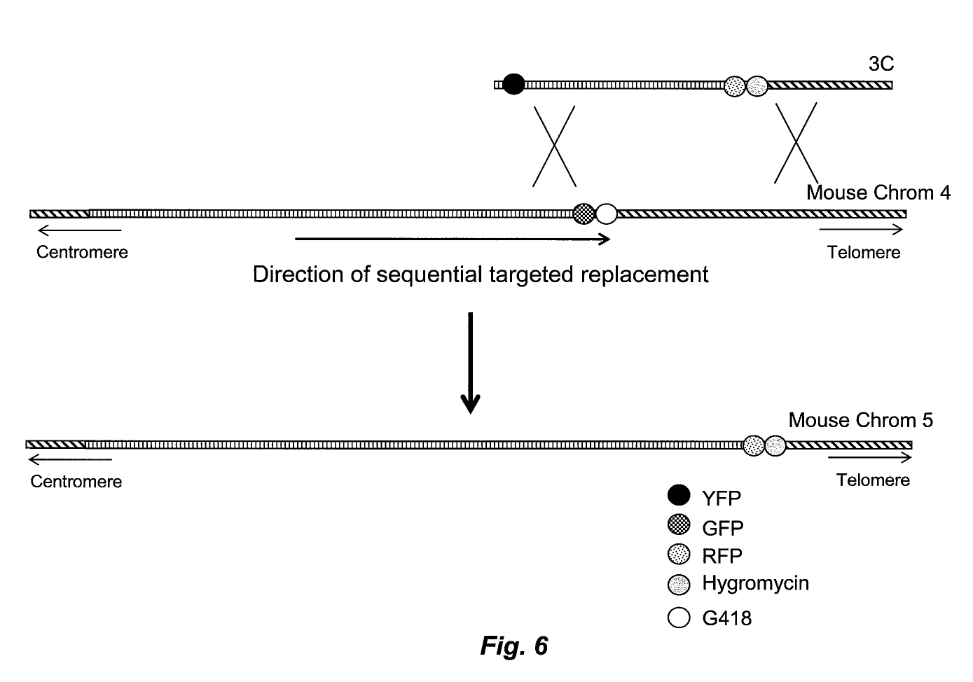

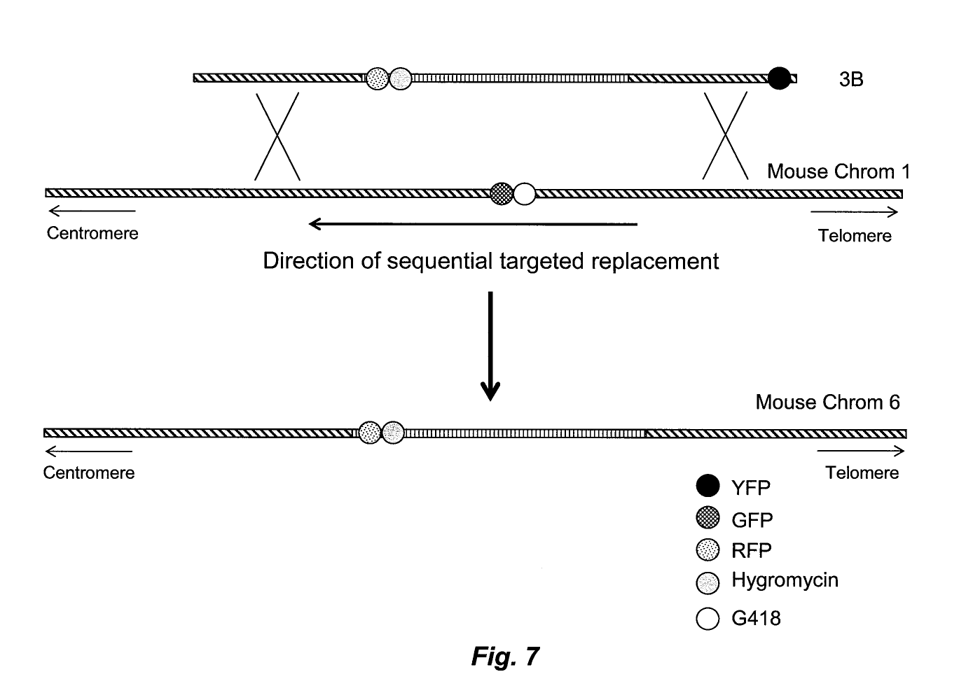

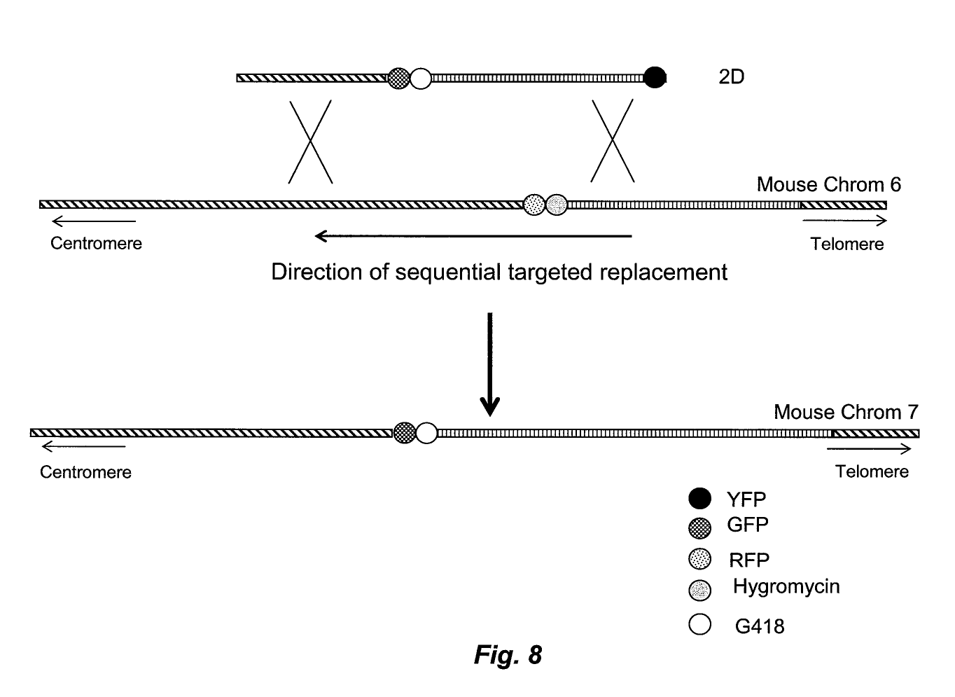

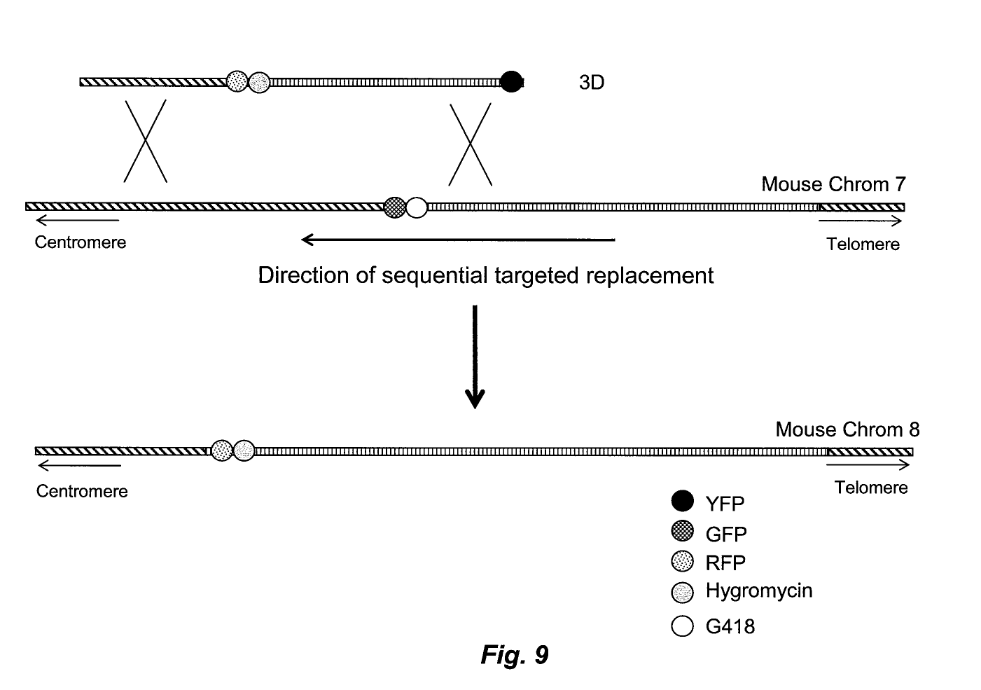

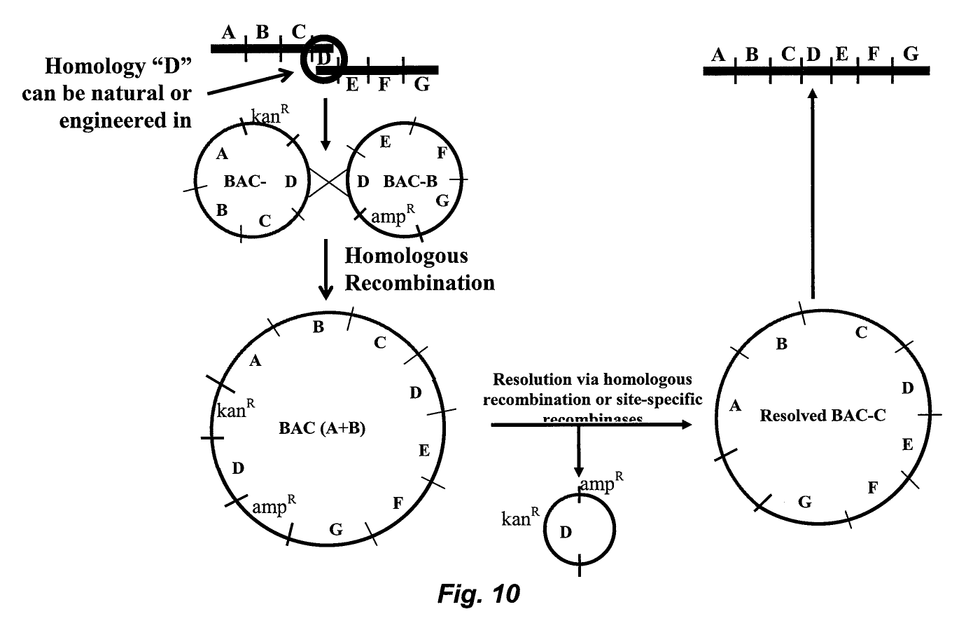

FIG. 5 illustrates homologous recombination between 1) a target chromosome containing the selection markers of construct 2B of FIGS. 3 and 2) a DNA construct equivalent to construct 1C (for this illustration, identified as 2C), that has GFP and G418 selection markers. The selection markers of the target mouse chromosome, RFP and hygromycin, are removed by the insertion of the incoming DNA construct that comprises GFP and G418 for markers. Note that as the additional construct is homologously recombined, the target sequence is incrementally replaced toward the telomere (i.e., direction of sequential targeted replacement).

FIG. 6 illustrates an extension of the sequential targeted replacement shown in FIG. 5. Another DNA construct equivalent to construct 1C (for this illustration, identified as 3C) continues the incremental addition of sequences toward the direction of the telomere, until the target DNA sequence is replaced by human sequences.

FIG. 7 illustrates sequential targeted replacement in the telomere to centromere direction. For this illustration, the DNA construct is equivalent to construct 1B (identified as 3B), and sequences of the target chromosome have been previously replaced with a construct (e.g., a 1A construct) having selection markers GFP and G418. Construct 3B homologously recombines with the target sequence, removes the previous selection markers, and introduces RFP and hygromycin.

FIG. 8 illustrates homologous recombination between 1) a target chromosome resulting from the recombination depicted in FIG. 7 (Mouse Chrom 6), and a DNA construct equivalent to construct 1D (for this illustration, identified as 2D) where the selection markers are removed by the incoming DNA construct, which comprises markers for GFP and G418. Note that as the additional construct is homologously recombined, the target sequence is incrementally replaced toward the centromere (i.e., direction of sequential targeted replacement).

FIG. 9 illustrates an extension of the sequential targeted replacement shown in FIG. 8, where a separate DNA construct equivalent to construct 1D (for this illustration, identified as 3D) continues the incremental addition of sequences in the direction of the centromere until the target DNA sequence is replaced by human sequences.

FIG. 10 illustrates homologous recombination of two BACs in E. coli. BAC-A carries DNA segments A-D and a kanamycin resistance gene. BAC-B carries DNA segments D-G and an ampicilin resistance gene. Following resolution, the recombined BAC (BAC-C) carries the contiguous DNA segments A-G.

### DETAILED DESCRIPTION

### Overview

A new approach is described herein to replace large DNA sequences with DNA sequences of a different species by homologous recombination. The method of the present invention is simpler than previous approaches, providing for the use of simpler qualitative procedures to assess targeted insertion of exogenous DNA into a host genome. Specifically, it provides for marker displacement in sequential homologous recombination steps, thereby allowing for the differentiation of cells containing randomly inserted sequences from those undergoing homologous recombination. This makes the process easier to employ while allowing for precise replacement of large DNA fragments.

The sequential transgenic replacement of genes in homologous recombination competent cells prepared by the method of the present invention provides the following advantages over prior methods: proper tissue specific expression, proper expression of alternative isoform expression because of faithful gene splicing, proper regulation of expression, physiological levels of expression, precise integration site, removal of the endogenous coding region, gene splicing and the production of in situ engineered DNA of about 50 kb and larger by incremental addition of cloning vector constructs, e.g., artificial chromosomes such as bacterial artificial chromosomes (BACs), of 1-350 kb or larger, for example, greater than about 1 kb, 10 kb, 50 kb, 100 kb, 200 kb, 300 kb, 350 kb and larger, which is limited primarily by the size of the coding region and the size of the incoming/overlapping vector, e.g., BAC. Other compatible systems include the use of DNA constructs that are derived from the DNA of P1 bacteriophage (PACs). PAC vectors can carry about 100 to 300 kb. YACs, yeast artificial chromosomes, may also be used if the YAC DNA is purified from other yeast chromosomes prior to introduction into the target homologous recombination competent cells.

In one embodiment of the system as disclosed, very large genes (e.g., the IgH locus in humans is well over one million base pairs, which is too large for one BAC) can be assembled by sequentially replacing contiguous regions of orthologous very large genes via successive BAC transfers in appropriate cells. The present invention allows for creation of a cell with 150 kb or more of a human gene, for example, then creation of a subsequent cell with transfer of the next 150 kb or more and so on.

The cells and organisms of the present invention can possess any one of multiple combinations of inserted genes. In one embodiment, the organism has a human gene coding sequence in place of an orthologous endogenous gene coding sequence. In another embodiment, the human coding sequence also includes gene expression regulatory (control) regions, such that the organism possesses both human control and human coding regions for the gene. In another embodiment, the humanized organisms have a human gene regulatory (control) region in place of an orthologous endogenous gene regulatory (control) region, but retain the endogenous coding region.

Additionally, the artificial chromosome system (e.g. BACs) as disclosed allows expression of multiple exogenous genes in a host. For example, one could potentially express human IgH and IgL as well as the genes for proteins with which they interact to regulate the antibody-based immune response and even further expanding to include genes for the T-cell based immune response, all in the same animal. As such, the invention allows addition of multiple DNA sequences using multiple BACs. As a consequence, partial or entire gene networks could be inserted into the genome of mice, for example. Entire gene clusters or multiple gene pathways, such as human metabolic pathways, heavy and light chain immunoglobulins, and the like, either with or without their associated human cis- and/or trans-acting regulatory sequences, can be expressed in an animal host with multiple human genes. Insertion of gene networks or clusters with “normal” coordinated tissue and inducible expression is not practicable with other transgenic technologies. For example, using the methods of the present invention, sequential genes could be added to an embryonic stem (ES) cell line that could be used to create a genetically engineered animal. Alternatively, genetically engineered animals could be made with ES cell lines containing one or more of the desired genes and then cross bred with other genetically engineered animals containing additional desired network or cluster genes made using the same processes of the invention.

### Definitions

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an” and “the” include plural references unless the context clearly dictates otherwise. Thus, for example, references to “a” or “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

“Polypeptide”, “peptide” and “protein” are used interchangeably to describe a chain of amino acids that are linked together by chemical bonds. For example, a polypeptide or protein may include immunoglobulin molecules and fluorescent proteins.

“Polynucleotide” refers to a chain of nucleic acids that are linked together by chemical bonds. Polynucleotides include, but are not limited to, DNA, cDNA, RNA, mRNA, and gene sequences and segments.

“Locus” refers to a location on a chromosome that comprises one or more genes, such as an IgH locus, the cis regulatory elements, and the binding regions to which trans-acting factors bind. As used herein, “gene” or “gene segment” refers to the coding region of a polynucleotide sequence encoding a specific polypeptide or portion thereof.

The term “endogenous” or “endogenous sequence” refers to a sequence that occurs naturally within the cell or organism. In certain embodiments, “endogenous sequence” refers to the DNA sequence that is endogenous for the final host cell or organism, including processes to design DNA constructs in another cell type or organism, such as E. coll. “Exogenous” or “non-endogenous sequence” refers to a polynucleotide which is not naturally present within the cell or organism. In certain embodiments, non-endogenous sequence may refer to a sequence present in the genome of the cell or organism that is introduced at a different locus or an alternate allele or mutated segment. “Orthologous sequence” refers to a polynucleotide sequence that encodes the corresponding polypeptide in another species, i.e. a human T-cell receptor and a mouse T-cell receptor. The term “syngeneic” refers to a polynucleotide sequence that is found within the same species that may be introduced into an animal of that same species, i.e. a mouse Ig gene segment introduced into a mouse Ig locus.

As used herein, the term “homologous” or “homologous sequence” refers to a polynucleotide sequence that has a highly similar sequence, or high percent identity (e.g. 30%, 40%, 50%, 60%, 70%, 80%, 90% or more), to another polynucleotide sequence or segment thereof. For example, a DNA construct of the invention may comprise a sequence that is homologous to a portion of an endogenous DNA sequence to facilitate recombination at that specific location. Homologous recombination may take place in prokaryotic and eukaryotic cells, and it may occur between two endogenous DNA sequences, two exogenous DNA sequences, or an endogenous and an exogenous DNA sequence.

As used herein, “flanking sequence” or “flanking DNA sequence” refers to a DNA sequence adjacent to the non-endogenous DNA sequence in a DNA construct that is homologous to an endogenous DNA sequence or a previously recombined non-endogenous sequence, or a portion thereof. DNA constructs of the invention may have one or more flanking sequences, e.g., a flanking sequence on the 3′ and 5′ end of the non-endogenous sequence or a flanking sequence on the 3′ or the 5′ end of the non-endogenous sequence.

The term “sequential replacement” refers to a series of homologous recombination steps, or events, to supplant or change one sequence of nucleotides from one source with a sequence of nucleotides from another source. For example, by using sequential replacement as disclosed in the present invention, an immunoglobulin locus from a non-human animal can be supplanted or replaced with a homologous immunoglobulin locus from a human.

As used herein, “target sequence” or “target DNA sequence” refers to the segment of the endogenous DNA sequence to be replaced during homologous recombination. The target sequence may be a locus, gene, or a portion thereof. For example, the full or entire target sequence to be replaced may be a polynucleotide sequence encoding a fragment of a polypeptide. In other embodiments, the target sequence may be a non-coding polynucleotide sequence.

The phrase “homologous recombination-competent cell” refers to a cell that is capable of homologously recombining DNA fragments that contain regions of overlapping homology. Examples of homologous recombination-competent cells include, but are not limited to, induced pluripotent stem cells, hematopoietic stem cells, bacteria, yeast, various cell lines and embryonic stem (ES) cells.

The term “non-human organism” refers to prokaryotes and eukaryotes, including plants and animals. Plants of the invention include, but are not limited to, corn, soy and wheat. Non-human animals include, but are not limited to, insects, birds, reptiles and mammals.

“Non-human mammal” refers to an animal other than humans that belongs to the class Mammalia. Examples of non-human mammals include, but are not limited to, non-human primates, rodents, bovines, ovines, equines, dogs, cats, goats, sheep, dolphins, bats, rabbits, and marsupials. In particular embodiments, the preferred non-human mammals are mice.

The terms “knock-in”, “genetically engineered” and “transgenic” refer to a cell or organism comprising a polynucleotide sequence, e.g., a transgene, derived from another species incorporated into its genome. For example, a mouse which contains a human H chain gene segment integrated into its genome outside the endogenous mouse IgH locus and a mouse which contains a human H chain gene segment integrated into its genome replacing an endogenous mouse H chain gene segment in the endogenous mouse IgH locus are both knock-in or transgenic mice. In knock-in cells and non-human organisms, the polynucleotide sequence derived from another species, may replace the corresponding, or orthologous, endogenous sequence originally found in the cell or non-human organism.

A “humanized” animal, as used herein refers to a non-human animal, e.g., a mouse, that has a composite genetic structure that retains gene sequences of the mouse or other nonhuman animal, in addition to one or more gene and or gene regulatory sequences of the original genetic makeup having been replaced with analogous human sequences.

As used herein, the term “vector” refers to a nucleic acid molecule into which another nucleic acid fragment can be integrated without loss of the vector's ability to replicate. Vectors may originate from a virus, a plasmid or the cell of a higher organism. Vectors are utilized to introduce foreign DNA into a host cell, wherein the vector is replicated. The term “vector DNA” refers to a DNA sequence adjacent to a DNA sequence homologous to a target endogenous sequence and/or a non-endogenous DNA sequence.

The term “bacterial artificial chromosome” or “BAC” as used herein refers to a bacterial DNA vector. In certain preferred embodiments the invention provides a BAC cloning system. BACs, such as those derived from E. coli, may be utilized for introducing, deleting or replacing DNA sequences of non-human cells or organisms via homologous recombination. The vector, pBAC, based on the E. coli single-copy plasmid F-factor can maintain complex genomic DNA as large as 350 kb and even larger in the form of BACs (see Shizuya and Kouros-Mehr, Keio J Med. 2001, 50(1):26-30). Analysis and characterization of thousands of BACs indicate that BACs are much more stable than cosmids or yeast artificial chromosomes (YACs). Further, evidence suggests that BAC clones represent the human genome far more accurately than cosmids or YACs. BACs are described in further detail in U.S. application Ser. No. 10/659,034, which is hereby incorporated by reference in its entirety. Because of this capacity and stability of genomic DNA in E. coli, BACs are now widely used by many scientists in sequencing efforts as well as in studies in genomics and functional genomics.

The term “construct” as used herein refers to a sequence of DNA artificially constructed by genetic engineering or recombineering. In one embodiment, the DNA constructs are linearized prior to recombination. In a preferred embodiment, the DNA constructs are not linearized prior to recombination.

As used herein, “selectable marker” or “selection marker” refers to an indicator that identifies cells that have undergone homologous recombination, and thereby allows for their selection. A DNA vector utilized in the methods of the invention can contain positive and negative selection markers. Positive and negative markers can be genes that when expressed confer antibiotic resistance to cells expressing these genes, for example, hygromycin resistance. Suitable selection markers can include, but are not limited to, Km (Kanamycin resistance gene), tetA (tetracycline resistance gene), G418 (neomycin resistance gene), van (vancomycin resistance gene), tet (tetracycline resistance gene), ampicillin (ampicillin resistance gene), methicillin (methicillin resistance gene), penicillin (penicillin resistance gene), oxacillin (oxacillin resistance gene), erythromycin (erythromycin resistance gene), linezolid (linezolid resistance gene), puromycin (puromycin resistance gene) and hygromycin (hygromycin resistance gene). The selection markers also can be metabolic genes that can convert a substance into a toxic substance. For example, the gene thymidine kinase when expressed converts the drug gancyclovir into a toxic product. Thus, treatment of cells with gancylcovir can negatively select for genes that do not express thymidine kinase. In a related aspect, the selection markers can be “screenable markers,” such as green fluorescent protein (GFP), yellow fluorescent protein (YFP), red fluorescent protein (RFP), GFP-like proteins, and luciferase. Such screenable markers can also be ectopically expressed markers, such as CD4, from the same or different species of the host cell, wherein the marker is not normally expressed in the host cell, such as embryonic stem cells, and the ectopic expression of the marker can be detected using fluorescence-based cell sorting.

### DNA Constructs

Exemplary DNA constructs of the invention contain an exogenous

DNA sequence, one or more DNA sequences homologous to the endogenous target DNA sequence and one or more sequences encoding selectable markers in a suitable vector. Various types of vectors are available in the art and include, but are not limited to, bacterial, viral, and yeast vectors. The DNA vector can be any suitable DNA vector, including a plasmid, BAC, YAC or PAC. In certain embodiments, the DNA vector is a BAC. Exemplary BACs of the invention include, but are not limited to: pBAC108L (ATCC Accession No. U511140) and pBeloBAC11 (ATCC Accession No. U51113).

The various DNA vectors are selected as appropriate for the size of DNA inserted in the construct. In one embodiment, the DNA constructs are bacterial artificial chromosomes or fragments thereof.

A polynucleotide sequence, e.g., the non-endogenous DNA sequence, can be contained in a vector, which can facilitate manipulation of the polynucleotide, including introduction of the polynucleotide into a target cell. The vector can be a cloning vector, which is useful for maintaining the polynucleotide, or can be an expression vector that contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, alpha virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb 25:37-42, 1993; Kirshenbaum et al., J. Clin. Invest 92:381-387, 1993; each of which is incorporated herein by reference).

In certain embodiments, a DNA construct of the invention is designed, or engineered, using homologous recombination in a bacterial cell, such as E. coli, prior to isolating the construct for transformation or transfection of the host cell or organism. For example, E. coli is transformed with a BAC containing the host (i.e., endogenous) target locus or a portion thereof. The BAC containing E. coli is then transformed with a recombination vector comprising the desired exogenous DNA sequence linked to 5′ and 3′ flanking sequences that mediate homologous recombination and cross-over between the exogenous sequence on the recombination vector and the endogenous sequence on the BAC.

Detection of homologously recombined BACs may utilize selectable markers incorporated into the vector. For example, when the second construct contains a selection marker, E. coli cells containing unrecombined vectors can be eliminated. BACs containing the non-endogenous sequence can be readily isolated from the bacteria and used for producing transgenic cells and organisms.

### Non-endogenous Sequence

The non-endogenous, or exogenous, DNA sequence of a DNA construct of the invention is the DNA sequence that will replace all or a portion of the target DNA sequence in the final host cell or organism. The non-endogenous DNA sequence may comprise only coding and/or include non-coding gene segments. As used herein, “gene” can refer to a wild-type allele (including naturally occurring polymorphisms) and mutant or engineered alleles. The genes utilized in the invention may be, for example, gene coding sequences or gene regulatory regions.

In certain embodiments, the non-endogenous sequence is mammalian. In another embodiment, the non-endogenous sequence is a human DNA sequence comprising all or a fragment of a gene. In still another embodiment, the non-endogenous DNA sequence is a human gene sequence encoding a human gene, having at least one intron contained therein.

The human DNA sequence to be used can be a human genomic sequence or can be a non-natural sequence encoding a human gene product. In one embodiment, the sequence is a non-natural sequence that encodes a human gene product, but has been codon-optimized for improved expression in the non-human animal. In another embodiment, the sequence is a chimeric gene that incorporates certain human exons but retains some non-human exons. In still another embodiment, the sequence is a chimeric gene that has some or all human exons, but keeps some or all non-human introns. In still another embodiment, the sequence is a chimeric gene that has some or all human exons, but keeps some or all non-human cis-regulatory elements in operable linkage with the human exons.

Human gene sequences utilized in the invention may include, but are not limited to, genes encoding G-protein coupled receptors, kinases, phosphatases, ion channels, nuclear receptors, oncogenes, cancer suppressor genes, viral and bacterial receptors, P450 genes, insulin receptors, immunoglobulins, metabolic pathway genes, transcription factors, hormone receptors, cytokines, cell signaling pathway genes and cell cycle genes. For example, specific human gene sequences include CD45, phenylalanine hydroxylase, factor VIII, cystic fibrosis transmembrane conductance regulator, NF1, utrophin, T-cell receptors, major histocompatibility complex, dystrophin, etc. In a preferred embodiment, the human gene encodes an immunoglobulin, or a fragment thereof.

Immunoglobulins are proteins produced by plasma cells that mediate the humoral immune response by binding to substances in the body that are recognized as foreign antigens. Each immunoglobulin unit is made up of two heavy chains (IgH) and two light chains (IgL) and has two antigen-binding sites. Immunoglobulins are grouped by structure and activity. The IgH constant region determines the isotype of the antibody, and the five classes, or isotypes, of immunoglobulins are IgA, IgD, IgE, IgG and IgM. There are two types of IgL, Igκ and Igλ.

### Endogenous Sequence

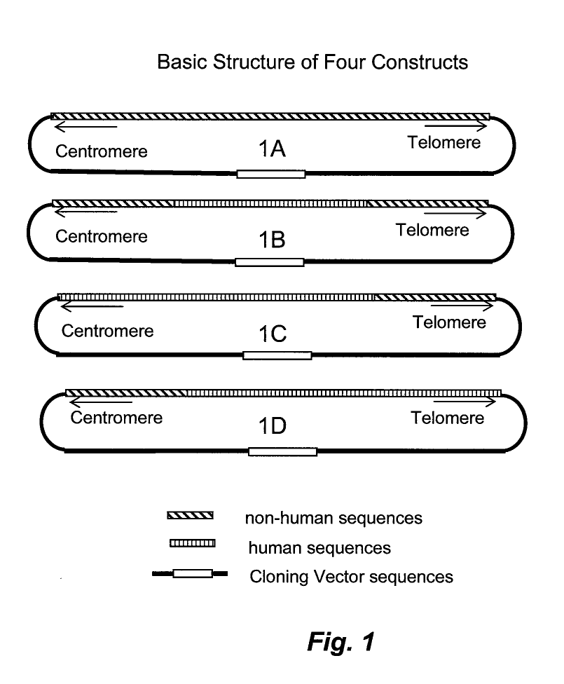

The endogenous flanking sequences are homologous to sequences in the genome of the host that flank the target DNA sequence. The DNA constructs of the invention may contain one or more endogenous flanking sequences on either side of the non-endogenous sequences (FIG. 1). For example, the construct may contain a first and a second endogenous DNA sequence flanking the non-endogenous DNA.

The regions flanking the non-endogenous DNA sequences utilized in the invention should be a length that allows for homologous recombination. For example, in certain embodiments each endogenous flanking DNA sequence for the first non-endogenous sequence is less than about 20 kb in length. For example, the flanking regions may be from about 0.1 to 19 kb, and typically from about 1 or 2 kb to 10 to 15 kb. In other embodiments, the flanking sequence length is greater than 20 kb in length.

Additionally, or alternatively, the constructs of the present invention contain non-endogenous sequences that are not flanked by endogenous sequences, which may be at either end of the construct. In certain embodiments, the DNA construct contains an endogenous sequence flanking one side of the non-endogenous sequence, i.e., on the 3′ end or the 5′ end of the non-endogenous DNA sequence. In a related embodiment, the non-endogenous sequence contains a segment that is homologous to a segment of a previously recombined non-endogenous segment, wherein the homologous non-endogenous sequences recombine and the single flanking endogenous sequence recombines with the homologous target sequence.

The methods of the invention can be used to precisely establish the joints between the non-endogenous and endogenous sequences. In one embodiment, only the endogenous coding sequence is replaced. In such an embodiment, the first endogenous DNA sequence in the second construct is joined at the 5′ of a start codon of the non-endogenous gene coding sequence and the second endogenous DNA sequence in the second construct is joined to the 3′ of a stop codon of the non-endogenous gene coding sequence. In another embodiment, only the endogenous regulatory (control) sequence is replaced. In still another embodiment, both the endogenous coding and regulatory (control) sequences are replaced.

In certain embodiments, the exogenous sequence is a human DNA sequence and the flanking sequences are non-human DNA sequences homologous to the host genome. In one embodiment, the non-human sequences are joined to the human sequence outside the coding region and including some or all of the 5′ and 3′ regulatory or control DNA sequences, including for example, promoter and enhancer sequences. Therefore, the non-human sequences can be joined to the human sequence adjacent to the 5′ end of the start codon or adjacent to the 3′ end of the stop codon. In one embodiment of the invention, a first DNA vector is constructed that has human DNA flanked by non-human DNA operably linked to only one end of the human DNA.

In a particular embodiment, the non-endogenous DNA sequence is a human sequence, and the one or more endogenous flanking sequences are mouse DNA segments. In this example, the host organism is a mouse, and the human DNA replaces a target sequence within the mouse genome upon homologous recombination. In certain embodiments, the mouse target sequence is an orthologous sequence.

### Target Sequence

The target sequence is the DNA sequence of the host genome that is to be specifically replaced upon homologous recombination. In specific embodiments, the target sequence is an orthologous DNA sequence. For example, a human gene encoding a cell surface receptor replaces the orthologous mouse cell surface receptor gene upon homologous recombination.

In other embodiments, the target sequence is not an orthologous DNA sequence. The target sequence may be chosen based on desired qualities of the locus into which it is to be introduced, including, but not limited to, expression level, homozygous viability, and chromosomal stability. For example, if a chosen non-endogenous sequence encodes a protein product to be isolated following expression, a chromosomal location having a high expression level may be used as the target sequence to be replaced.

### Selection Markers

DNA constructs of the invention contain one or more sequences encoding selection, or selectable, markers for use in indentifying cells that have successfully undergone homologous recombination and incorporated the non-endogenous DNA sequence. The markers may be positive or negative selection markers. Selection markers include antibiotic resistance genes, fluorescent proteins, ectopic proteins, and metabolic genes.

For example, a DNA construct is cloned in a BAC or P1 bacteriophage (PAC) vector, and includes sequences encoding one or more of YFP, GFP, RFP, G418 and hygromycin resistance. In particular embodiments, the DNA construct contains at least two selection markers. In another aspect, one of the selection markers is a fluorescent marker.

The DNA construct of the present invention may carry positive and/or negative selection markers that can interrupt the non-endogenous or endogenous DNA sequence. The vectors can be engineered such that one intron can have a selection marker encoded within the intron. When a selection marker is included, clones undergoing a desired recombination event may be selected using an appropriate antibiotic or drug or identifying a fluorescent protein, etc.

Additional selection markers may be added following the recombining step to the recombined construct. In one embodiment, a selection marker is added within an intron in the non-endogenous DNA sequence. In yet another embodiment, a selection marker is added to a position flanking an endogenous DNA sequence.

In certain embodiments, the non-endogenous sequence is human DNA and the endogenous flanking sequences are non-human DNA. In one embodiment, a human/non-human DNA construct comprises a first and second selection marker, wherein the first and second selection markers are adjacent to each other within the human or non-human region of a DNA construct. In one aspect, the first and second selection markers are contained entirely within the human region of a DNA construct or entirely within the non-human region of a DNA construct. In another aspect, the first and second selection markers are at or near the junction between the human and non-human region(s) of a DNA construct.

The placement of the first and second selection markers on the human/non-human construct (e.g., 2C, of FIG. 5) should be chosen such that they are within the boundaries of where recombination is to take place; i.e., within the region bounded by the crosses (X) in the Figures. For example, if the first and second adjacent selection markers lie outside of the bounded regions, the first and second adjacent selection markers on the construct will not recombine appropriately with the chromosomal target.

In one aspect, the first and second adjacent selection markers are contained on a human/non-human construct, where a separate third selection marker is positioned distal to the first and second adjacent selection markers, where the position of the third distal selection marker is opposite and centromeric or opposite and telomeric relative to the position of the first and second adjacent selection markers (see FIGS. 2-9). For example, if the first and second adjacent selection markers are positioned toward the 3′ end of the sense strand on a construct, where the 3′ end is directed toward the centromere, the third distal selection marker is positioned 5′ distal on the sense strand, toward the telomere. Conversely, if the first and second adjacent selection markers are positioned toward the 5′ end of the sense strand on a construct, where the 5′ end is directed toward the telomere, the third distal selection marker is positioned 3′ distal on the sense strand, toward the centromere. In addition, if the first and second adjacent selection markers are in the middle of the human/non-human DNA construct, the third distal selection marker may be at either end. Further, the third distal selection marker lies outside of the region bounded by the crosses (X) in the Figures and functions as a negative selection marker.

### Sequential Targeted Replacement

Following the recombination steps in a bacterial cell as described above, a set of recombined DNA constructs can be isolated, the constructs having the various sequences and orientations as described. The constructs can then be introduced sequentially into a homologous recombination competent cell, thereby replacing the endogenous target sequence. Contacting cells with DNA constructs may involve steps such as transforming, transfecting, electroporating, or microinjecting.

In addition, if the constructs were engineered in E. coli with the DNA components required for chromosome function, e.g., telomeres and a centromere, preferably, but not required, of the recipient species (i.e. host or endogenous species) for optimal function, e.g., mouse telomeres and a mouse centromere, they can be introduced into the recipient cell by electroporation, microinjection etc. and would function as artificial chromosomes. These constructs also may be used as a foundation for subsequent rounds of homologous recombination for building up larger and larger artificial chromosomes.

The invention provides a method for replacing an endogenous target DNA sequence in a cell with a non-endogenous DNA sequence using one or more DNA constructs, such that cell comprises the non-endogenous sequence, i.e., transgene, following a series of homologous recombination steps. While all types of DNA constructs are contemplated by the invention, BACs are presented herein as a prototypical example. For example, a cell is contacted with a first BAC containing a non-endogenous sequence flanked by homologous endogenous sequences and a first set of one or more selection markers. Cells that have undergone a successful recombination are identified using the selection markers and confirmed using further qualitative means such as Southern blots of restriction digested genomic DNA using a probe just outside the boundary of one of the flanking regions to detect restriction fragment length polymorphisms created when the non-endogenous DNA sequence replaced the endogenous DNA.

In certain embodiments, recombined cells are then contacted with a second BAC containing a non-endogenous sequence that contains an overlapping sequence homologous to the non-endogenous sequence of the first construct at one end and an endogenous flanking sequence at the opposite end along with a second set of one or more selection markers. In particular embodiments, the non-endogenous sequence in the cell is extended as more of the target sequence is replaced during homologous recombination. In addition, the first set of selection markers may be removed when the second set is introduced into the cell. Cells that have incorporated the second set of markers can then be identified and isolated. The homologous recombination and selection steps are repeated with additional BACs until the target DNA sequence is replaced. The consecutive BACs may either alternate selection marker sets or contain new selection markers on each BAC, so that following each sequential recombination event, a new set of selection markers can be utilized to identify cells which have incorporated the non-endogenous DNA sequence.

For example, cells containing fluorescent markers, such as GFP, RFP and YFP, can be identified using flow cytometry, fluorescence assisted cell sorting (FACS), or fluorescence microscopy. Upon identification, recombined cells are then isolated for further expansion or for the generation of a transgenic organism. Further to the identification of selection makers, methods of confirming a successful homologous recombination event include, but are not limited to, Southern blots, restriction fragment length polymorphism (RFLP) analysis, fluorescence in situ hybridization (FISH), and PCR.

In an illustrative example, the invention provides a method of generating a cell containing a transgene, the method involving recombining a first DNA construct including DNA sequences homologous to target DNA sequences, one or more sequences encoding one or more selection markers, and cloning vector DNA; a second DNA construct including DNA sequences to replace endogenous targeted DNA sequences, flanking DNA sequences homologous to endogenous sequences in the cell to be transformed or transfected, one or more sequences encoding one or more selection markers, and cloning vector DNA; and a third and fourth DNA construct including two DNA sequences, one or more sequences encoding one or more selection markers, and cloning vector DNA.

In one aspect, the first DNA construct of the set of constructs serves as a substrate sequence for homologous recombination with endogenous DNA sequences present in target cells. In a related aspect, the second DNA construct of the set of constructs serves as both a substrate sequence for homologous recombination and a replacement sequence of DNA in the cells. In one aspect, a third and/or fourth DNA construct comprises a single endogenous flanking sequence. In another aspect, the third and/or fourth DNA construct does not comprise flanking sequences.

The invention also provides a DNA construct for performing homologous recombination within a cell, having a human DNA coding sequence with at least one intron and one or more selection marker genes contained within the at least one intron. In one embodiment, recombination in a cell directs replacement of the non-human gene with its human ortholog.

### Transgenic Organisms

Transgenic organisms generated from recombined cells identified by selection markers include both plants and animals. Transgenic animals of the invention include, but are not limited to, insects, birds, reptiles, and non-human mammals. In particular embodiments, the non-human mammal is a mouse.

After engineering the non-endogenous sequence into homologous recombination-competent cells to replace portions or all of the endogenous target sequence, genetically engineered non-human animals, such as mice, can be produced by now-standard methods such as blastocyst microinjection followed by breeding of chimeric animals, morula aggregation or cloning methodologies, such as somatic cell nuclear transfer. In some cases, animals produced by these methods will be further bred to produce homozygous animals.

For animals for which there is a current lack of ES cell technology for genetic engineering through blastocyst microinjection or morula aggregation, the endogenous loci can be modified in cells amenable to various cloning technologies or developmental reprogramming (e.g., induced pluripotent stem cells, IPS). The increased frequency of homologous recombination provided by the BAC technology provides the ability to find doubly replaced loci in the cells, and cloned animals derived therefrom would be homozygous for the mutation, therein saving time and costs especially when breeding large animals with long generation times. Iterative replacements in cultured cells could provide all of the requisite engineering at multiple loci and allowing for direct production of animals using cloning or IPS technology without cross-breeding to get the appropriate genotypes. The ability to finely tailor the introduced non-endogenous sequences and also finely specify the sites into which they are introduced provides the ability to engineer enhancements that would provide better function.

In one illustrative example, ES cells from a non-human animal can be selected for recombinants by including positive and/or negative selection markers in the recombined DNA vector. The ES cells are then introduced into a blastocyst of a non-human animal or the ES cells are allowed to divide and observed for the presence of the marker. If the former, the chimeric blastocyst may then be introduced into a pseudopregnant host animal to generate a humanized non-human animal. Other methods for generating embryos from ES cells also can be used with the methods of the invention. However, the first transfected ES cells may be transfected again until the entire target gene is replaced, then introduced into a blastocyst.

The methods of the invention can be used with any homologous recombination competent cells from any non-human animal. In one embodiment, the cells are mouse ES cells and the non-human animal is a mouse, and the methods of the invention are used to create a humanized mouse. Prior to generation of the humanized mouse, for example, sequentially replacing contiguous regions of very large orthologous genes by successive BAC transfers in progeny cells by the present invention allows for creation of a cell with 350 Kb or more of the human gene, then creation of a subsequent cell with transfer of the next 350 Kb and so on.

Furthermore, the system as disclosed has flexibility. One can, through cross-breeding, introduce additional genes modified according to the invention to the transgenic animals. For example, to engineer mice that make humanized antibodies, both the endogenous immunoglobulin heavy chain (IgH) and a least one of the endogenous immunoglobulin light chain loci, either kappa (Igk) or lambda (Igl), and preferably both, would need to be replaced with a portion or all of their human orthologues. The engineering of the loci could be accomplished in separate projects using ES cells and genetically-engineered mice derived therefrom then cross-bred to obtain progeny with both humanized IgH and IgL loci. Later, other large gene complexes or multi-gene families important for regulation of the immune network such as the major-histocompatibility locus and the T-cell receptor locus or the FcγR multi-gene family could be humanized and the mice bred with mice having humanized Ig loci. Such mice would be useful for generating a human-like immune response for better human antibody-drug discovery. They would also provide a useful model system for testing of antibody-drug candidates for immunogenicity and activity, especially if the gene for the antigen (drug target) were also humanized in the same mice. Other gene pathways with complexly orchestrated regulation could be humanized in the same way. Besides utility for antibody drug development, an appropriately humanized animal would have a number of important uses for the pharmaceutical industry in drug development. Humanizing a drug-target gene in a mouse or other smaller species allows more rapid and less costly testing of biologic and small-molecule drugs for activity and toxicology because the drug will now bind to and modulate the human target rather than the heterologous target, which may have lower or zero binding affinity. Entire human drug metabolism pathways can be reconstituted in a mouse by replacing the mouse genes with their human orthologues, allowing faster and less-expensive absorption, distribution, metabolism and excretion toxicity (ADME-tox) testing. Entire disease pathways can also be reconstituted for target discovery and validation as well as drug discovery and validation.

### EXAMPLES

The following examples are provided as further illustrations and not limitations of the present invention.

### Example 1

### DNA CONSTRUCTS

To employ the approach of the present methods, four types of DNA constructs may be used. They may be chosen based on the specific needs of the gene replacement desired.

The first type of construct (1A in FIG. 1) has 1) DNA sequences homologous to endogenous DNA sequences, 2) one or more sequences that supply selection markers, and 3) cloning vector DNA sequences.

One may generate a DNA construct carrying an endogenous flanking sequence having genes for GFP and G418 resistance, cloned in a BAC vector, such as the pBeloBAC11 vector.

The second type of DNA construct (1B in FIG. 1) has 1) non-endogenous DNA sequences to replace endogenous target DNA sequences, 2) flanking DNA sequences homologous to endogenous sequences in the cell to be transformed or transfected, 3) sequences for one or more selection markers, and 4) cloning vector DNA sequences. In this way one can generate a DNA construct cloned in a BAC vector, having genes for RFP and Hygromycin resistance, and human sequences flanked by mouse sequences that are homologous to endogenous mouse sequences.

The third and fourth types of constructs (1C and 1D in FIG. 1) contain a non-endogenous DNA sequence, an endogenous DNA sequence, a gene or genes for selection markers, and cloning vector DNA. The endogenous DNA sequences (for example mouse sequences) of the constructs serve as substrate sequences for homologous recombination with endogenous DNA sequences present in target cells. The non-endogenous DNA sequences (for example human sequences) of the constructs serve as both substrate sequences for homologous recombination and replacement sequences of DNA in the cells. Therefore, unlike the second type of construct, the non-endogenous sequences of these two DNA constructs are flanked on only one side by a sequence that is homologous to the endogenous target sequence. Therefore, a DNA construct can be generated having 1) a human sequence, 2) a mouse sequence that is homologous to endogenous mouse sequences, 3) a gene or genes as selection markers, and 4) cloning vector DNA sequences for sequential homologous recombination events to elongate the non-endogenous DNA sequence in the cell.

As depicted in FIG. 1, the human sequences in constructs 1C and 1D are not flanked on both sides by the non-human sequences, and the human and non-human sequences are joined at adjacent positions. The two constructs differ in the relative order of the two sequences. The order is determined by the direction of consecutive replacement of existing sequences in the cells with replacing DNA sequences. For example, during the sequential replacement process, if the direction of consecutive replacement is from centromere to telomere in the cells, the DNA construct has the human sequences at the centromere side and the mouse sequences are at the telomere side (1C in FIG. 1). If the intended direction is from telomere to centromere in the cells, the DNA construct has the human sequences at the telomere side and the mouse sequences at the centromere side (1D in FIG. 1).

### Example 2

### HOMOLOGOUS RECOMBINATION OF BACs IN E. COLI

The DNA constructs of the invention may be designed and cloned in vectors such as BACs. Homologous recombination in E. coli can be used to construct BACs with larger inserts of DNA than is represented by the average size of inserts of currently available BAC libraries. Such larger inserts can comprise DNA representing a human locus, or a portion thereof.

A BAC vector is based on the F-factor found in E. coli. The F-factor and the BAC vector derived from it are maintained as low copy plasmids, generally found as one or two copies per cell depending upon its life cycle. Both F-factor and BAC vector show the fi⁺ phenotype that excludes an additional copy of the plasmid in the cell. By this mechanism, when E. coli already carries and maintains one BAC, and then an additional BAC is introduced into the E. coli, the cell maintains only one BAC, either the BAC previously existing in the cell or the external BAC newly introduced. This feature is extremely useful for selectively isolating BACs homologously recombined as described below.

The homologous recombination in E. coli requires the functional RecA gene product. In this example, the RecA gene has a temperature-sensitive mutation so that the RecA protein is only functional when the incubation temperature is below 37° C. When the incubation temperature is above 37° C., the Rec A protein is non-functional or has greatly reduced activity in its recombination. This temperature sensitive recombination allows manipulation of RecA function in E. coli so as to activate conditional homologous recombination only when it is desired. It is also possible to obtain, select or engineer cold-sensitive mutations of Rec A protein such that the protein is only functional above a certain temperature, e.g., 37° C. In that condition, the E. coli would be grown at a lower temperature, albeit with a slower generation time, and recombination would be triggered by incubating at above 37° C. for a short period of time to allow only a short interval of recombination.

Homologous recombination in E. coli is carried out by providing overlapping DNA substrates that are found in two circular BACs. The first BAC (BAC-A) carries the contiguous segments from A through D, and the second BAC (BAC-B) carries the contiguous segments from D through G (FIG. 10). The segment D carried by both BACs is the overlapping segment where the DNA crossover occurs, and as a result it produces a recombinant that carries the contiguous segments from A through G.

BAC-A described above is the one already present in the cell, and when BAC-B is introduced into the cell, either BAC-A or BAC-B can exist in the cell, not both BACs. Upon electroporation of BAC-B into the cell, the temperature would be lowered below 37° C. so as to permit conditional RecA activity, therein mediating homologous recombination. If BAC-A and BAC-B have a selectable marker each and the markers are distinctively different, for example, BAC-A carries Kan (a gene conferring kanamycin resistance) and BAC-B carries Amp (a gene giving Ampicilin resistance), only the recombinant BAC grows in the presence of both antibiotics Kan and Amp. The resolution is accomplished by homologous recombination between shared homology in the two vector sequences. Alternatively, sites for site-specific recombinases such as loxP/CRE or frt/flp can be employed to introduce site-specific recombinase recognition sequences into the vector sequences, either BAC-A or BAC-B, and then when the site-specific recombinase is expressed or introduced, recombination will occur between the sequences, therein deleting the vector sequences and the duplicated segment D. Upon deletion of vector sequences during resolution, one or both of the selection markers may also be deleted. However, resolution may also be accomplished without deleting the selection markers. The resolved BAC has now the contiguous stretch from A through G with single copy of D (see BAC-C in FIG. 10).

The introduction of a BAC to E. coli cell is typically done by electroporation. Prior to electroporation, the cells are maintained at 40° C., a non-permissive temperature for recombination, and after electroporation the cells are incubated at 30° C., a temperature permissive for recombination. During the incubation, homologous recombination occurs and cells express enzymes necessary to become resistant to both antibiotics. The incubation period is about 45 to 90 minutes. Then the cells are spread on the media plates containing both antibiotics and the plates are incubated at 40° C. to prevent further homologous recombination. The majority of colony isolates growing on the media plates have the recombined BAC that has predicted size. This can be confirmed by pulsed field gel electrophoresis analysis.

### Example 3

### ISOLATION OF BAGS AND INTRODUCTION INTO EUKARYOTIC CELLS

In preparation for introduction into homologous recombination competent cells, such as ES cells, expression cassettes can be recombined onto the DNA constructs, e.g., BACs. For example, mammalian cassettes carry genes with required regulatory elements such as promoters, enhancers and poly-adenylation sites for expression of the genes in mammalian cells, such as mouse ES cells. The genes on the cassette include selectable markers used to select and screen for cells into which the BAC has been introduced and homologously recombined.

For introduction into homologous recombination competent eukaryotic cells, BAC DNA is purified from E. coli and the E. coli genomic DNA by methods known in the art such as the alkaline lysis method, commercial DNA purification kits, CsCl density gradient, sucrose gradient, or agarose gel electrophoresis, which may be followed by treatment with agarase. The purified DNA may then be linearized by methods known in the art, e.g., NotI, AscI, AsiSI, FseI, PacI, PmeI, SbfI, and SwaI digestion. The circular or linearized DNA, typically 0.1-10 μg of DNA depending upon the size of the construct, is introduced into the eukaryotic cells, such as ES cells, by methods known in the art such as transfection, lipofection, electroporation, calcium precipitation or direct nuclear microinjection.

### Example 4

### SEQUENTIAL REPLACEMENT OF A TARGET SEQUENCE IN EUKARYOTIC CELLS

The first BAC to be introduced into eukaryotic cells may be comprised of a DNA sequence homologous to the corresponding endogenous genome and one or more selection sequences. Homologous recombination in the cells results in the incorporation of selection markers in the host genome (FIG. 2). The selection markers contained on this first BAC, e.g., GFP and G418, can be utilized as negative selection markers following the next homologous recombination event when the next BAC contains a sequence which will replace the endogenous sequence containing the first set of selection markers (FIG. 3).

Alternatively, the first BAC (or the second BAC following the first BAC described above) to be introduced into eukaryotic cells may be comprised of exogenous DNA flanked on either side by 1 kb to 10 kb to 100 kb or more of endogenous DNA from the corresponding endogenous genome in the cells. The first BAC then replaces a portion of the endogenous genome by homologous recombination in the cells, replacing the endogenous DNA between the two flanking DNAs, i.e., the target sequence, with the exogenous DNA engineered between the flanking DNAs on the BAC (FIG. 4).

For example, by constructing in E. coli a BAC that contains 300 kb of a human DNA sequence flanked on the 3′ end by mouse DNA corresponding to the region 3′ of the mouse target sequence and flanked 5′ by mouse DNA corresponding to the region 300 kb 5′ of the target mouse sequence, and introducing the purified BAC into mouse ES cells to allow for homologous recombination, the corresponding mouse DNA sequence would be replaced by the orthologous human DNA. The flanking mouse DNAs could also be further away, e.g., the 5′ homology could be further upstream of the endogenous target sequence so that upon homologous recombination, most or the entirety of the mouse locus would be replaced by the human sequence on the BAC. In other words, the length of the region of the endogenous DNA to be replaced is dictated by the distance between the two flanking mouse segments on the BAC. The distance is not the actual length between the mouse segments in the BAC; rather it is the distance between the mouse segments in the endogenous mouse chromosome. This distance may be calculated from the available genomic databases, such as UCSC Genomic Bioinformatics, NCBI and others known in the art.

Any subsequent, BAC would have two segments flanking the DNA to be introduced. Of the two flanking DNA sequences, one is comprised of non-endogenous DNA that corresponds to all or a portion of the non-endogenous DNA introduced into the cell genome in the first replacement and the other is endogenous DNA corresponding to endogenous DNA upstream (or downstream as the case may be) of the region to be replaced in the second introduction.

Upon introduction into a homologous recombination-competent cell such as a mouse ES cell into which non-endogenous DNA from a previously introduced BAC has replaced a portion of the endogenous locus, one crossover would occur between the non-endogenous flanking sequence of the BAC and the non-endogenous sequence in the modified host chromosome, and the other between the endogenous flanking sequence of the BAC and the homologous region of the endogenous chromosome (FIGS. 5-9).

In this way, when they are joined by homologous recombination in cells, the joined segments become a contiguous germline-configured segment as it is naturally found in the organism of origin for the non-endogenous sequence. This process is repeated with subsequent BACs until all of the desired target replacement is completed.

### Example 5

### REPLACEMENT OF A TARGET SEQUENCE FROM THE 5′ DIRECTION

The direction of the replacement in homologous recombination—competent cells, such as ES cells, may be performed either from the 5′ end or 3′ end of the transcriptional direction. However, BAC modification should be done according to the configuration of the homology requirement for homologous recombination in competent cells.

For example, in the 5′ end direction, the first BAC to be used has the telomere side of the non-endogenous sequence, flanked on either side by homologous endogenous DNA for targeting into the endogenous locus (FIG. 7). The subsequent BACs to be used in the iterative replacement process is a BAC modified as described above having non-endogenous sequences replacing endogenous sequences in part of or all of the endogenous target locus (FIGS. 8 and 9). The DNA upstream of the endogenous germline configured DNA would be non-endogenous DNA corresponding to a portion already integrated into the modified locus and the downstream DNA would be the endogenous sequence 3′ of the target sequence. As noted above, the flanking DNAs may range in size from 1 kb to 10 kb to 100 kb to larger.

### Example 6

### REPLACEMENT OF A TARGET SEQUENCE FROM THE 3′ DIRECTION

In the 3′ direction, the first BAC is a modified BAC based on the first BAC for the 5′ directional replacement in that the first BAC has the centromere side of the non-endogenous DNA sequence (FIG. 3). The subsequent BACs are modified BACs of the BACs used for the replacement from the 5′ direction. The modification is that the endogenous flanking DNA is located at the opposite end of the non-endogenous sequence, e.g., the telomere side (FIGS. 5 and 6).

### Example 7

### SELECTION OF CELLS FOLLOWING HOMOLOGOUS RECOMBINATION

In order to detect and identify cells containing targeted recombinants resulting from successful homologous recombination events, i.e., existing and/or endogenous sequences are replaced with incoming sequences, selection markers are included in the constructs. Selection markers are a group of genes encoding fluorescent proteins, drug resistance genes or genes that confer other forms of selectivity, for example, genes that result in ectopic expression of any identifiable marker (e.g., surface expression of a xenogeneic protein or a protein not expressed by the cell type). The incorporation of these markers allows for the identification of recombined cells by using qualitative assays.

Cells expressing the fluorescent proteins are detected by a fluorescent microscope, FACS, or any other equipment capable of detecting fluorescence emitted from the proteins. Cells harboring drug resistant genes are able to grow in the presence of the drugs. Other markers are detected by tagged antibodies, or color presentation.

The selection marker encoding sequences are placed on one or both flanking homologous sequences to the endogenous region and/or on the replacing sequences. The locations of selection marker genes in the construct are strategically determined according to the point where DNA crossover between incoming and endogenous DNA occur. Positive selection markers are internal to the flanking targeting DNA so as to be stably integrated into the genome along with the replacing DNA. Thus, markers for positive selection are located within the region of crossover, while negative markers lie outside of this region (see YFP placement in FIGS. 2-9).

Optimally, the BAC would carry a screenable marker such as GFP or RFP approximately adjacent to another selection marker such as hygromycin resistance or G418. GFP⁺ or RFP⁺ cells could be detected by FACS or fluorescence microscopy.

To confirm homologous recombination of selected cells, genomic DNA is recovered and restriction fragment length polymorphism (RFLP) analysis performed by a technique such as Southern blotting with a DNA probe from the endogenous loci, said probe mapping outside the replaced region. RFLP analysis shows allelic differences between the two alleles, the endogenous DNA and incoming DNA, when the homologous recombination occurs via introduction of a novel restriction site in the replacing DNA. Because the flanking DNA arms may be large and difficult to resolve by standard agarose gel electrophoresis, low percentage agarose gels may be used or CHEF gel electrophoresis may be used. Alternatively, a restriction site may be purposely engineered into the replacing DNA on the BAC during the engineering in E. coli so as to engineer a conveniently sized fragment spanning the junction of the introduced DNA and the endogenous DNA upon restriction digest, and encompassing the designated probe sequence.

For engineering subsequent BACs, different selectable markers are just internal to one flanking arm while the opposite flanking arm for homologous recombination, which overlaps with the flanking arm carrying the selection markers used in targeting the BAC, carries no markers, such that the homologous recombination event deletes the markers introduced in targeting BAC and introduces a new selection marker at the opposite end (internal from the opposite flanking arm). For example, fluorescent markers alternate between GFP and RFP after each round of homologous recombination occurs such that round 1 introduces GFP and round 2 deletes GFP and introduces RFP. If random insertion occurs, both fluorescent markers exist in the cells. A flow cytometer with cell sorting capability can be utilized to sort and retain cells based on the presence of signals from one fluorescent protein and the absence of signal from another.

Drug resistance markers can be used similarly except that in most cases simultaneous dual selection (resistance for one drug and sensitivity to another is not possible) with the exception of HPRT and thymidine kinase selections. Otherwise, clones would be picked and duplicate plates made, one to test for drug resistance and one to test for drug sensitivity.

In either dual drug-selection testing or dual fluorescent marker screening, the assays are qualitative in nature. Through standard advanced planning it is possible to replace endogenous DNA with non-endogenous DNA across megabase-sized loci through iterative rounds of homologous recombination using only 2 different pairs of combinations of one selectable marker and one screenable marker. However, three or more sets each of selectable and screenable markers could also be used.

For example, upon transfection of the constructs (2B in FIG. 3) to cells that already have reporter genes from a previous replacement by targeted homologous recombination (Mouse Chrom 2 in FIG. 3), if the entire or nearly entire construct is randomly inserted into one or more sites outside the targeted regions, both sets of selection markers, those already present and those that are incoming, will be expressed in the cells. However, if targeted homologous recombination occurs, the selection marker genes from the incoming construct replace the previous reporters, which are subsequently removed due to DNA strand exchange between the incoming construct and existing chromosome (Mouse Chrom 3 in FIG. 3). Thus, to find such cells, marker sets of the already existing and incoming constructs must be different, i.e., they must contain either different fluorescent proteins or different drug resistance genes. For example, if the mouse chromosome has a selection marker set of RFP and Hygromycin (Mouse Chrom 3 in FIG. 5), then the incoming marker set is GFP and G418 (2C in FIG. 5). Random insertion of the incoming construct results in cells that show both green and red fluorescence and G418 and Hygromycin resistance, whereas cells having the construct inserted at the targeted site show only green fluorescence and G418 resistance (Mouse Chrom 4 in FIG. 5). This process is alternatively repeated with different selection markers until all of the desired targeted replacement is completed.

In order to enrich for cells having homologously targeting events, one selection marker (YFP in FIGS. 2 to 9) can be placed outside the targeted region of the construct in such a way so that when homologous recombination occurs in ES cells, the marker is lost from the recombinant. To eliminate non-transformants of ES cells (cells not having integrated constructs regardless of their locations), selection markers of G418 and Hygromycin resistance genes are used.

According to methods of the invention, the final engineered chromosome will retain the selection marker(s) at one terminus, depending upon the direction of iterative replacement. The marker(s) can be engineered to be flanked by loxP or frt sites in the BAC engineering in E. coli. Subsequently, expression of Cre or flp recombinase, respectively, in either the cells or the genetically engineered organism derived therefrom will trigger site-specific recombination between the loxP or frt sites, thereby deleting the marker(s).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of sequentially replacing a non-endogenous DNA sequence across a target non-human DNA sequence comprising:
a) contacting a cell, wherein the cell comprises the target non-human DNA sequence, with a first DNA construct and homologously recombining the first DNA construct with the target non-human DNA sequence, where the first DNA construct comprises,
i) a first non-endogenous DNA sequence flanked by a first and a second non-human DNA sequence, and
ii) a first selection marker sequence;
b) qualitatively determining the presence of the first selection marker in the cell, thereby identifying a first selection marker positive cell having a first recombined target non-human DNA sequence therein;
c) contacting the first selection marker positive cell with a second DNA construct and homologously recombining the second DNA construct with the recombined target non-human DNA sequence including the first non-endogenous DNA sequence, wherein the second DNA construct comprises,
i) a second non-endogenous DNA sequence operably linked to a third non-human DNA sequence, wherein the second non-endogenous DNA sequence homologously recombines with a segment of the first non-endogenous DNA sequence of the recombined target non-human DNA sequence, and wherein the third non-human DNA sequence homologously recombines with non-human DNA sequences distal to the second non-human DNA sequence of the first DNA construct, and
ii) a second selection marker sequence, wherein the second marker sequence is contained within the third non-human DNA sequence, and wherein the first and second selection markers are not the same; and
d) qualitatively determining the presence of the second selection marker in a second cell, said second cell comprising the recombined target non-human DNA sequence of step (c), wherein homologous recombination at step (c) removes the first selection marker sequence, thereby identifying a second selection marker positive cell;
wherein the target non-human DNA sequence is replaced by the non-endogenous DNA sequence.

2. The method of claim 1, further comprising:
e) contacting the second selection marker positive cell with a third DNA construct and homologously recombining the third DNA construct with the recombined target non-human DNA sequence of step (d) comprising the first and second non-endogenous DNA sequences, wherein the third DNA construct comprises,
i) a third non-endogenous DNA sequence operably linked to a fourth non-human DNA sequence, wherein the third non-endogenous DNA sequence homologously recombines with a segment of the second non-endogenous DNA sequence of the recombined target non-human DNA sequence, and wherein the fourth non-human DNA sequence homologously recombines with non-human DNA sequences distal to the third non-human DNA sequence of the second DNA construct, and
ii) a third selection marker sequence, wherein the third marker sequence is contained within the fourth non-human DNA sequence; and
f) qualitatively determining the presence of the third selection marker in a third cell, said third cell comprising the recombined target non-human DNA sequence of step (e), where homologous recombination at step (e) removes the second selection marker sequence, thereby identifying a third selection marker positive cell.

3. The method of claim 2, further comprising:
g) repeating steps (c)-(f), where each added DNA construct includes,
i) a non-endogenous DNA sequence, wherein the non-endogenous DNA sequence recombines with a segment of the previously recombined non-endogenous DNA sequence of the previous DNA construct, a non-human DNA sequence, wherein the non-human DNA sequence homologously recombines with non-human DNA sequences distal to the non-endogenous and target non-human DNA sequences of the previously recombined DNA construct, and
ii) a selection marker sequence, wherein recombination of the additional DNA construct alternately removes the previous selection marker sequence,
wherein step (g) is repeated until the target non-human DNA sequence is replaced by the non-endogenous DNA sequence.

4. The method of claim 2, wherein the first and third selection marker sequences encode the same selection marker.

5. The method of claim 1, wherein the second non-endogenous DNA sequence replaces a portion of the target DNA sequence 5′ of the first recombined non-endogenous DNA sequence, thereby replacing the target DNA sequence in the 3′ to 5′ direction.

6. The method of claim 1, wherein the second non-endogenous DNA sequence replaces a portion of the target DNA sequence 3′ of the first recombined non-endogenous DNA sequence, thereby replacing the target DNA sequence in the 5′ to 3′ direction.

7. The method of claim 1, wherein the first and second non-human DNA sequences in step (a)(i) are greater than or equal to 20 kb in length.

8. The method of claim 1, wherein the first and second non-human DNA sequences in step (a)(i) are less than about 20 kb in length.

9. The method of claim 1, wherein the non-endogenous DNA sequence is orthologous to the target non-human DNA sequence.

10. The method of claim 1, wherein the non-endogenous DNA sequence is a human DNA sequence.

11. The method of claim 1, wherein the cell is a plant cell.

12. The method of claim 1, wherein the cell is a non-human animal cell.

13. The method of claim 12, wherein the non-human animal cell is a mouse embryonic stem cell.

14. The method of claim 1, wherein the selection marker is a fluorescent marker.

15. The method of claim 1, wherein the selection marker is a drug resistance marker.

16. The method of claim 1 further comprising a second selection marker, wherein the second selection marker is adjacent to the first selection marker.

17. The method of claim 16, wherein one of the selection markers is a fluorescent marker.

18. The method of claim 16, wherein one of the selection markers is a drug resistance marker.

19. The method of claim 16, wherein the first selection marker is a fluorescent marker, and the second selection marker is a drug resistance marker.

20. A set of DNA constructs comprising:
a) a first DNA construct comprising sequences homologous to a target DNA sequence, a selection marker sequence, and a cloning vector DNA sequence; and
b) a second DNA construct comprising a non-endogenous sequence for homologous replacement of a target DNA sequence, flanking DNA sequences homologous to an endogenous sequence in a target cell, a selection marker sequence, and a cloning vector DNA sequence.

21. The set of claim 20, further comprising a third DNA construct comprising a non-endogenous DNA sequence, a DNA sequence homologous to an endogenous sequence in the target cell, a selection marker sequence, and a cloning vector DNA sequence.

22. The set of claim 21 further comprising a fourth DNA construct comprising a non-endogenous DNA sequence, a DNA sequence homologous to the target sequence, a selection marker sequence, and a cloning vector DNA sequence.

23. The set of claim 20, wherein the DNA sequences of the first DNA construct serve as substrate sequences for homologous recombination with endogenous DNA sequences present in target cells.

24. The set of claim 20, wherein the DNA sequences of the second DNA construct serve as both substrate sequences for homologous recombination and replacement sequences of DNA in the cells.

25. The set of claim 20, wherein the selection marker is a fluorescent marker.

26. The set of claim 20, wherein the selection marker is a drug resistance marker.

27. The set of claim 26, further comprising a fluorescent marker.

28. The set of claim 20, wherein the selection marker is placed within the coding region of the non-endogenous or non-human DNA sequence.

29. The set of claim 20, wherein the selection marker is placed within the non-coding region of the non-endogenous or non-human DNA sequence.

30. The set of claim 20, wherein each DNA construct is cloned in a vector.

31. The set of claim 30, wherein the vector is a BAC, YAC or PAC vector.

32. A non-human cell comprising a transgene generated by the method of claim 1.

33. A non-human animal generated from the cell of claim 32.

34. A humanized mouse comprising a transgene generated by the method of claim 10.

35. A method of producing a recombined BAC comprising:
a) contacting a bacterial cell, wherein the bacterial cell comprises a first BAC, with a second BAC, wherein said first BAC comprises a first non-endogenous DNA sequence, a first selection marker sequence and a cloning vector DNA sequence; and wherein said second BAC comprises a second non-endogenous DNA sequence, a second selection marker sequence and a cloning vector DNA sequence; wherein said second non-endogenous DNA sequence comprises an overlapping segment of said first non-endogenous DNA sequence; wherein homologous recombination occurs at said overlapping segment; and
b) qualitatively determining the presence of said first and second selection markers in the bacterial cell having a recombined non-endogenous DNA sequence,
wherein the recombined BAC is produced.

36. The method of claim 35, further comprising resolving said recombined BAC, wherein the overlapping segment is removed from the BAC, thereby generating a resolved BAC.

37. The method of claim 36, wherein the first selection marker is removed from said recombined BAC.

38. The method of claim 36, wherein the second selection marker is removed from said recombined BAC.

39. The method of claim 36, wherein the first and second selection markers are removed from said recombined BAC.

40. The method of claim 36, wherein said resolving comprises homologous recombination.

41. The method of claim 36, wherein said resolving comprises a site-specific recombinase.

42. The method of claim 41, wherein said site-specific recombinase is Cre.

43. The method of claim 41, wherein said site-specific recombinase is flp.

44. The method of claim 35, wherein said first selection marker is a drug resistance marker.

45. The method of claim 35, wherein said first selection marker is a fluorescent marker.

46. The method of claim 35, wherein said second selection marker is a drug resistance marker.

47. The method of claim 35, wherein said second selection marker is a fluorescent marker.

48. The method of claim 35, wherein said first and second selection markers are drug resistance markers.

49. A recombined BAC produced according to the method of claim 35.

50. A resolved BAC generated according to the method of claim 36.

51. A set of BACs comprising:
a) a first BAC comprising a first non-endogenous DNA sequence, a first selection marker sequence, and a cloning vector DNA sequence; and
b) a second BAC comprising a second non-endogenous DNA sequence and a second selection marker sequence, wherein said second non-endogenous DNA sequence comprises an overlapping region of said first non-endogenous DNA sequence, wherein homologous recombination occurs at said overlapping region.

52. The set of claim 51, wherein said first selection marker sequence is a fluorescent marker.

53. The set of claim 51, wherein said first selection marker sequence is a drug resistance marker.

54. The set of claim 51, wherein said second selection marker is a fluorescent marker.

55. The set of claim 51, wherein said second selection marker is a drug resistance marker.
